(19) United States
(12) Patent Application Publication
(10) Pub. No.: US 2024/0318119 A1
(43) Pub. Date: Sep. 26, 2024

(54) METHOD AND DEVICE FOR REGULATING TEMPERATURE OF INCUBATOR, AND INCUBATOR

(71) Applicants: QINGDAO HAIER BIOMEDICAL TECHNOLOGY CO., LTD., Qingdao (CN); QINGDAO HAIER BIOMEDICAL CO., LTD., Qingdao (CN)

(72) Inventors: Huan CHEN, Qingdao (CN); Wei HU, Qingdao (CN); Zepeng DUAN, Qingdao (CN); Zhihong XU, Qingdao (CN); Huanwen JU, Qingdao (CN); Panfei WANG, Qingdao (CN); Dongchen YU, Qingdao (CN); Haitao CHEN, Qingdao (CN)

(73) Assignees: QINGDAO HAIER BIOMEDICAL TECHNOLOGY CO., LTD., Qingdao (CN); QINGDAO HAIER BIOMEDICAL CO., LTD., Qingdao (CN)

(21) Appl. No.: 18/560,086

(22) PCT Filed: Aug. 2, 2022
(86) PCT No.: PCT/CN2022/109715

§ 371 Date: Nov. 9, 2023

(30) Foreign Application Priority Data
Sep. 9, 2021 (CN) 202111058144.9

(51) Int. Cl. C12M 1/02; C12M 1/00; G05D 23/19; H05B 1/02

(52) U.S. Cl. CPC C12M 41/24; C12M 41/14; G05D 23/1917; H05B 1/023

(57) ABSTRACT

Disclosed are a method and a device for regulating a temperature of an incubator, an inner surface of the incubator being arranged with heating wires. The method includes: in case where a door body of the incubator is opened and then reclosed, obtaining a set temperature of the incubator; according to at least one of a heating power corresponding to the set temperature or and a temperature range corresponding to the set temperature, determining a target heating power; and controlling the heating wires to operate at the target heating power to adjust the internal temperature of the incubator to the set temperature.

### CROSS-REFERENCE TO RELATED APPLICATIONS

This application is the National Stage of International Application No. PCT/CN2022/109715, filed on Aug. 2, 2022, which claims priority to Chinese Patent Application No. CN202111058144.9, filed on Sep. 9, 2021. All of the aforementioned applications are incorporated herein by reference in their entireties.

### TECHNICAL FIELD

The present disclosure relates to the technical field of temperature control, for example, relates to a method and a device for regulating the temperature of an incubator, and an incubator.

### BACKGROUND

The incubator is a box-shaped device mainly for cultivating microorganisms. This device is widely used in experiments such as constant temperature cultivation and constant temperature reaction. Specifically, it is a device for the vitro cultivation of microorganisms by simulating to form a similar environment for the growth of the microorganisms in the incubator. It is an advanced instrument for microorganism cultivation and further makes the requirements of this equipment extremely strict in temperature, humidity, and internal environment.

The conventional temperature control methods for the incubator, usually via one or more sets of PID (Proportion, Integration, Differentiation) algorithms, control the power of one or more paths of the heating wires in the incubator, in order to achieve temperature control of the incubator. However, in practical operation, after a period of operation of the incubator, when the operator performs the action of opening or closing the door, the temperature inside the incubator will dissipate to the outside, resulting in a decrease in the internal temperature of the incubator. After the operator closes the door body of the incubator, it is difficult to achieve rapid warming in a case where the incubator controls the heating of the heating wires based on the PID algorithm, which leads to the inability of the internal temperature of the incubator rapidly restoring to the set temperature and affects the cultivation effect of microorganisms in the incubator.

### SUMMARY

In order to have a basic understanding of some aspects of the disclosed embodiments, a brief summary is given below. The summary is not intended to be a general comment to identify crucial/essential constituent elements or to describe the protection scope of these embodiments but rather to serve as a preface to the detailed description that follows.

The embodiment of the present disclosure provides a method and a device for regulating temperature of an incubator, and an incubator, in order to achieve rapid warming after the operator closes the door body of the incubator and reduce the adverse impact on cultivation effect caused by opening or closing the door.

In some embodiments, the method comprises: in a case where the door body of the incubator is opened and then reclosed, obtaining a set temperature of the incubator, according to a heating power corresponding to the set temperature or a temperature range corresponding to the set temperature, determining a target heating power, and controlling the heating wire to operate at the target heating power to adjust an internal temperature of the incubator to the set temperature.

In some embodiments, the method comprises: according to a preset first correspondence, taking the heating power corresponding to the set temperature as the target heating power.

In some embodiments, the method comprises: according to a preset second correspondence, determining a target scale factor corresponding to the temperature range, and according to the target scale factor, calculating the target heating power.

In some embodiments, the step of according to the target scale factor, calculating the target heating power comprises: P_{target}=Pₘₐₓ×(Qₙ−a×t), where P_{target} is the target heating power, Pₘₐₓ is a maximum heating power of the incubator, Qₙ is the target scale factor, a is an amplitude factor, and t is a heating cycle.

In some embodiments, the method comprises: in a case where the door body of the incubator is opened and then reclosed, obtaining a temperature change rate inside the incubator, and in a case where the temperature change rate inside the incubator is greater than or equal to a preset change rate, obtaining the set temperature of the incubator.

In some embodiments, the method comprises: in a case where the internal temperature of the incubator decreases and a magnitude thereof exceeds a preset threshold, readjusting the internal temperature of the incubator to the set temperature of the incubator.

In some embodiments, the method comprises: determining a secondary heating power of the heating wire, and controlling the heating wire to operate at the secondary heating power.

In some embodiments, the method comprises: determining the secondary heating power by the following formula, P_{2nd}=Pₘₐₓ×(Qₘ−b×t), where P_{2nd} is the secondary heating power, Pₘₐₓ is a maximum heating power of the incubator, Qₘ is the secondary scale factor, b is a secondary amplitude factor, and t is a heating cycle.

In some embodiments, the device comprises: a processor and a memory storing program instructions, the processor being configured to execute the aforementioned method for regulating the temperature of the incubator when executing the program instructions.

In some embodiments, the incubator comprises: a heating wire arranged on the inner surface of the incubator; and the aforementioned device for regulating the temperature of the incubator.

The method and the device for regulating the temperature of an incubator, and an incubator provided by the embodiment of the disclosure can have the following technical advantages: by obtaining a set temperature of the incubator in a case where the door body of the incubator is opened and then reclosed, and combined with a heating power corresponding to the set temperature or a temperature range corresponding to the set temperature, determining a target heating power for regulating the internal temperature of the incubator. Thus, in the situation of opening or closing the door of the incubator, the heating wire of the incubator is controlled to operate at the determined target heating power, which replaces the conventional control methods of the heating wire with one or more sets of PID control algorithms. By means of mandatory intervention, the internal temperature of the incubator is rapidly restored to the set temperature of the incubator, so as to reduce the adverse impact on the cultivation effect caused by opening or closing the door of the incubator.

The above general description and the description below are exemplary and explanatory only and are not intended to limit the present disclosure.

### BRIEF DESCRIPTION OF THE DRAWINGS

One or more embodiments are illustrated by means of the corresponding drawings, which do not constitute a limitation of the embodiments, elements having the same reference numerals in the drawings are shown as similar elements, and the drawings do not constitute a limitation of proportion, where:

FIG. 1 is a schematic diagram of a method for regulating the temperature of an incubator provided by an embodiment of the present disclosure;

FIG. 2 is a schematic diagram of a method for calculating the target heating power provided by the embodiment of the present disclosure;

FIG. 3 is a schematic diagram of a method for obtaining the set temperature of an incubator provided by the embodiment of the present disclosure;

FIG. 4 is another schematic diagram of a method for regulating the temperature of an incubator provided by an embodiment of the present disclosure;

FIG. 5 is another schematic diagram of a method for regulating the temperature of an incubator provided by an embodiment of the present disclosure; and

FIG. 6 is a schematic diagram of a device for regulating the temperature of an incubator provided by an embodiment of the present disclosure.

### DETAILED DESCRIPTION OF THE EMBODIMENTS

In order to enable a more detailed understanding of the features and technical content of the embodiments of the present disclosure, the implementation of the embodiments of the present disclosure will be described in detail below in conjunction with the accompanying drawings, which are for illustration only and are not intended to limit the embodiments of the present disclosure. In the following technical description for convenience of explanation, several details are provided for a full understanding of the disclosed embodiments. However, one or more embodiments may still be practiced without these details. In other instances, the well-known structures and devices may simplify the disclosure in order to simplify the drawings.

The terms “first”, “second” and the like in the specification and claims of embodiments of the present disclosure and the above drawings are used to distinguish similar elements and are not necessarily used to describe a particular order or priority. It should be understood that the data used in this way can be interchanged where appropriate for the embodiments of the present disclosure described herein. Furthermore, the terms “comprise” and “have” and any variations thereof are intended to cover a non-exclusive inclusion.

Unless otherwise illustrated, the term “multiple” means two or more.

In the embodiment of the present disclosure, the character “/” indicates that the front element and rear element are in an “or” relationship. For example, A/B illustrates A or B.

The term “and/or” is an association relationship that describes elements, indicating that there can be three relationships. For example, “A and/or B” represents relationships: A or B, or A and B.

The term “corresponding to” can refer to an association or binding relationship, while A corresponding to B refers to an association or binding relationship between A and B.

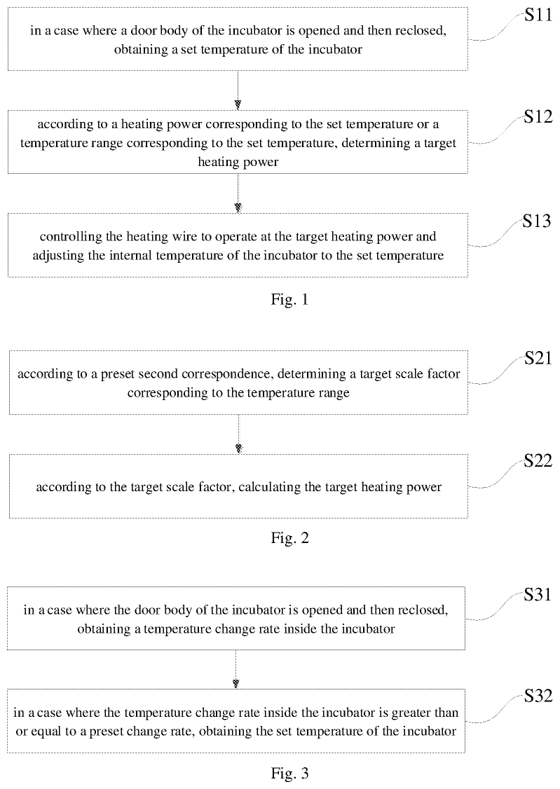

FIG. 1 is a schematic diagram of a method for regulating the temperature of an incubator provided by an embodiment of the present disclosure. With reference to FIG. 1, a method for regulating temperature of an incubator provided by the embodiment of the present disclosure comprises:
S11, in a case where a door body of the incubator is opened and then reclosed, obtaining a set temperature thereof;
S12, according to a heating power corresponding to the set temperature or the temperature range corresponding to the set temperature, determining the target heating power; and
S13, controlling a heating wire to operate at the target heating power to adjust an internal temperature of the incubator to the set temperature.

In the present solution, the incubator refers to a box-shaped device with the heating wire arranged on an inner surface for temperature, and is used for cultivating microorganisms and animal and plant cells. For example, a carbon dioxide incubator, etc. Specifically, the set temperature of the incubator can be obtained when an operator opens and then recloses the door body of the incubator. Here, the operator may be any laboratory technician or other personnel who can perform operations of opening or closing the incubator. The set temperature is the maintained temperature inside the incubator set by the operator based on the cultivated microbial information in the incubator. In one example, the set temperature of the incubator can be determined by display parameters on a display panel of the incubator, in this way, the set temperature of the incubator can be accurately obtained. Further, the target heating power for regulating the internal temperature of the incubator can be determined by combining the heating power corresponding to the determined set temperature or the temperature range corresponding to the determined set temperature. In this way, it is convenient for the incubator to control the heating wire to operate at the determined target heating power, in order to accurately regulate the internal temperature of the incubator.

The method and the device for regulating temperature of an incubator, and an incubator provided by the embodiment of the disclosure can have the following technical advantages: by obtaining a set temperature of the incubator in a case where the door body of the incubator is opened and then reclosed, and combining a heating power corresponding to the set temperature or a temperature range corresponding to the set temperature, to determining a target heating power used to regulate the internal temperature of the incubator. Thus, in the situation of opening and closing the door of the incubator, the heating wire of the incubator is controlled to operate at the determined target heating power, which replaces the conventional control methods of the heating wire with one or more sets of PID control algorithms. By means of mandatory intervention, the internal temperature of the incubator is regulated to rapidly restore to the set temperature of the incubator, so as to reduce the adverse impact on cultivation effect caused by opening or closing the door of the incubator.

Optionally, the step of S12, according to a heating power corresponding to the set temperature, determining the target heating power comprises:
according to a preset first correspondence, defining the heating power corresponding to the set temperature as the target heating power.

In the present solution, combined with the determined set temperature of the incubator, the target heating power of the heating wire on the inner surface of the incubator can be determined via various steps. In one example, the first correspondence can be stored in the incubator or a server associated with the incubator. Here, the first correspondence is the correspondence between the set temperature and the heating power. Specifically, the operator can obtain the first correspondence based on multiple experiments on the same culture material. In another example, the target heating power can be determined by combining the temperature range corresponding to the set temperature. Specifically, the target temperature range corresponding to the set temperature can be matched from multiple preset temperature ranges. In one example, three temperature ranges can be preset considering the required temperature of cell culture. It can be understood that the general set temperature of the incubator is 3° C. higher than the ambient temperature where the incubator is located. In one example, there are three preset temperature ranges comprising: the first temperature range Tₛₑₜ<38° C., the second temperature range 38° C.≤Tₛₑₜ<45° C., and the third temperature range 45° C.≤Tₛₑₜ<55° C. For example, if the set temperature is 39° C., the target temperature range corresponding to the set temperature is the second temperature range. Thus, combined with the matched second temperature range, the incubator determines the target heating power for regulating the internal temperature of the incubator. In this way, the target heating power of the heating wire in the incubator can be determined via various steps.

FIG. 2 is a schematic diagram of a method for calculating the target heating power provided by the other embodiment of the present disclosure. With reference to FIG. 2, optionally, the step of, according to a temperature range corresponding to the set temperature, the incubator determines the target heating power comprises:
S21, according to a preset second correspondence, determining the target scale factor corresponding to the temperature range; and
S22, according to the target scale factor, calculating the target heating power.

In the present solution, the second correspondence can be pre-stored. The second correspondence is the correspondence between the temperature range and a scale factor. It can be understood that the set temperature is positively proportional to the scale factor, that is, the higher the set temperature, the greater the corresponding scale factor, and the higher the corresponding temperature range. In one example, the correspondence between the temperature range and the scale factor comprises that a scale factor corresponding to the first temperature range is 0.8, a scale factor corresponding to the second temperature range is 0.9, and a scale factor corresponding to the third temperature range is 1. Specifically, after the incubator determines the temperature range corresponding to the set temperature, the target scale factor corresponding to the temperature range can be determined based on the pre-stored second correspondence, and the target heating power of the heating wire can be calculated by combining the target scale factor. In this way, effectively combined with the temperature ranges of different set temperatures, the more targeted scale factor is determined, so as to provide a more accurate data foundation for determining the target heating power of the heating wire.

Optionally, the step of, according to the target scale factor, calculating the target heating power comprises:

$${P}_{target} = {P}_{max} \times ( {Q}_{n} - a \times t )$$

where P_{target} is the target heating power, Pₘₐₓ is a maximum heating power of the incubator, Qₙ is the target scale factor, a is an amplitude factor, and t is a heating cycle.

In the present solution, after determining the target scale factor, different steps for calculating the target heating power can be determined by combining different target scale factors. Specifically, if the temperature range corresponding to the set temperature is the first temperature range, then the target heating power of the heating wire can be determined by P_{target}=Pₘₐₓ×(0.8−a×t). If the temperature range corresponding to the set temperature is the second temperature range, then the target heating power of the heating wire can be determined by P_{target}=Pₘₐₓ×(0.9−a×t). If the temperature range corresponding to the set temperature is the third temperature range, then the target heating power of the heating wire can be determined by P_{target}=Pₘₐₓ×(1−a×t). Here, Pₘₐₓ is the maximum heating power of the incubator, of which value is determinate and related to the volume of the incubator. In one example, if the volume of the incubator is 168 L, the corresponding Pₘₐₓ is 800 watts. The letter t represents the heating cycle, which can be preset by the operator. The heating wire has the same heating power within the same heating cycle. For example, if the heating cycle of the heating wire is 5 s, the corresponding target heating power of the heating wire changes every five seconds. The letter a is the amplitude factor, which is a constant value and can be defined as 0.02. In this way, effectively combined with different scale factors, the determining step of the target heating power corresponding to the set temperature is accurate, so that the incubator can obtain a more accurate target heating power via the determining step.

FIG. 3 is a schematic diagram of a method for obtaining the set temperature of an incubator provided by the other embodiment of the present disclosure. With reference to FIG. 3, optionally, the step of S11, in a case where the door body of the incubator is opened and then reclosed, obtaining the set temperature, comprises:
S31, in a case where the door body of the incubator is opened and then reclosed, obtaining the temperature change rate inside of the incubator; and
S32, in a case where the temperature change rate inside the incubator is greater than or equal to a preset change rate, obtaining the set temperature of the incubator.

In the present solution, the difference between the temperature inside the incubator when the door body of the incubator is opened and then reclosed and the temperature inside the incubator before the door body is opened is obtained, and the temperature change rate is determined as the ratio of the difference to the temperature inside the incubator before the door body is opened. Specifically, in order to accurately determine the timing of regulating temperature required for the incubator after the operator performs the action of opening or closing the door, in the present solution, the temperature change rate inside the incubator can be determined, and in a case where the temperature change rate of the incubator is greater than or equal to the preset change rate, the current incubator is determined to regulate the temperature and to obtain the set temperature of the incubator. The preset change rate can be set to 0.5%. In this way, the timing of regulating the temperature of the incubator is accurately determined, and regulating the temperature of the incubator is avoided in case there is no temperature change or low temperature change inside the incubator after the operator opens or closes the door of the incubator, and the incubator processing resources is effectively saved.

Optionally, the step of S11, in a case where the door body of the incubator is opened and then reclosed, obtaining the set temperature comprises:
in a case where the door body of the incubator is opened and then reclosed, obtaining an interval time between the door body being opened and the door being reclosed; and
in a case where the interval time is greater than or equal to the preset interval time, obtaining the set temperature.

In the present solution, the interval time between the door body being opened and the door body being reclosed can be obtained. Specifically, in order to accurately determine the timing of regulating temperature required for the incubator after the operator performs the action of opening or closing the door, the interval time between the operator performing the actions of opening the door body and closing the door body can be determined in the present solution. And in a case where the interval time is greater than or equal to the preset interval time, a current temperature loss in the incubator can be determined, the regulating temperature is required, and the set temperature of the incubator can be obtained. The preset interval time can be set as two minutes. In this way, the timing of regulating the temperature of the incubator is accurately determined, and regulating the temperature of the incubator is avoided in case there is no temperature change or low temperature change inside the incubator due to the short interval time between the door being opened and the door being reclosed by the operator, and the incubator processing resources is effectively saved.

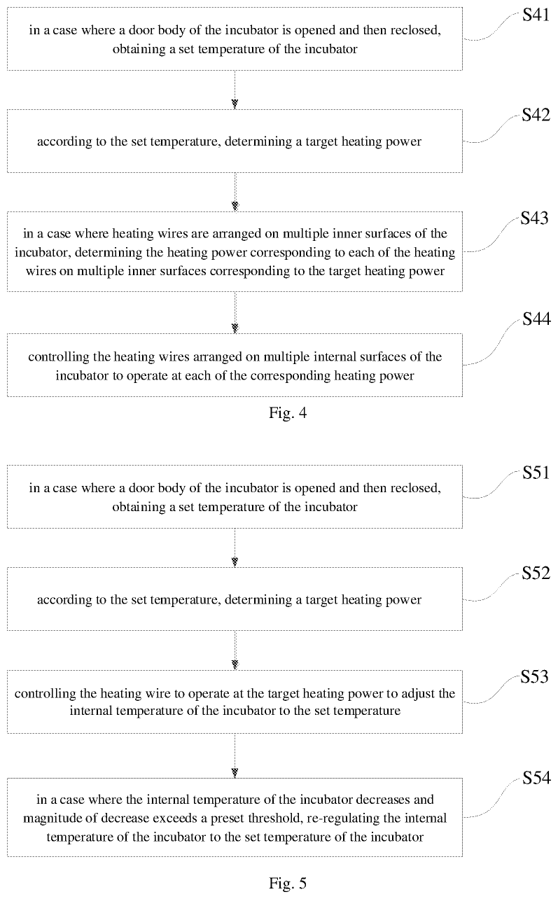

FIG. 4 is another schematic diagram of a method for regulating the temperature of an incubator provided by an embodiment of the present disclosure. The multiple inner surfaces of the incubator are arranged with the heating wires. With reference to FIG. 2, a method for regulating the temperature of an incubator provided by the embodiment of the present disclosure comprises:
S41: in a case where the door body of the incubator is opened and then reclosed, obtaining the set temperature;
S42: according to the set temperature, determining the target heating power;
S43: in a case where heating wires are arranged on multiple inner surfaces of the incubator, determining each of the corresponding heating power of the heating wires on multiple inner surfaces corresponding to the target heating power; and
S44: controlling the heating wires arranged on multiple inner surfaces of the incubator to operate at each of the corresponding heating power.

In the present solution, the incubator can be a carbon dioxide incubator. Specifically, multiple inner surfaces of the carbon dioxide incubator are arranged with the heating wires. In order to determine each of the corresponding heating power of the heating wire on multiple inner surfaces after determining the target heating power, the correspondence between the heating power and each of the corresponding heating power of the heating wire can be pre-stored. Furthermore, after determining the target heating power, each of the corresponding heating power of the heating wire arranged on multiple inner surfaces corresponding to the target heating power can be matched from the correspondence, to control the heating wires arranged on multiple inner surfaces of the incubator to operate at each of the corresponding heating power. With this solution, it can be effectively determined the each of the corresponding heating power of the heating wire arranged on multiple inner surfaces of the carbon dioxide incubator, so as to replace the conventional one set or multiple sets of control algorithms to control each heating wire, by means of mandatory intervention in each of the corresponding heating power of the heating wire, the internal temperature of the carbon dioxide incubator is regulated and controlled to rapidly restore to the set temperature of the carbon dioxide incubator, so as to reduce the adverse impact on cultivation effect caused by opening or closing the door of the carbon dioxide incubator.

In a preferable solution, after determining the target heating power, each of the corresponding heating power of the heating wire on each surface of the carbon dioxide incubator can also be determined based on the preset ratio relationship. In one example, the preset ratio relationship can be bottom:left:right:top:back:door body=4:1:1:2:2:2. For example, if the determined target heating power is 1000 W, the heating power of the heating wire on the bottom side of the carbon dioxide incubator is 330 W, the heating power of the heating wire on the left side of the carbon dioxide incubator is 83 W, the heating power of the heating wire on the right side of the carbon dioxide incubator is 83 W, the heating power of the heating wire on the top side of the carbon dioxide incubator is 167 W, the heating power of the heating wire on the back side of the carbon dioxide incubator is 167 W, and the heating power of the heating wire for the door body of the carbon dioxide incubator is 167 W. With this solution, the target heating power of the carbon dioxide incubator is more accurately allocated to each of the corresponding heating power of the heating wires on each surface. By accurately determining each heating power, the internal temperature of the carbon dioxide incubator is regulated, to effectively prevent condensation in the carbon dioxide incubator.

FIG. 5 is another schematic diagram of a method for regulating the temperature of an incubator provided by an embodiment of the present disclosure. With reference to FIG. 5 a method for regulating the temperature of the incubator provided by the embodiment of the present disclosure comprises:
S51: in a case where the door body of the incubator is opened and then reclosed, obtaining a set temperature of the incubator;
S52: according to the set temperature of the incubator, determining the target heating power;
S53: controlling the heating wire to operate at the target heating power to adjust the internal temperature of the incubator to the set temperature; and
S54: in a case where the internal temperature of the incubator decreases and a decreasing magnitude exceeds a preset threshold, re-regulating the internal temperature to the set temperature of the incubator.

In the present solution, after adjusting the internal temperature to the set temperature of the incubator, it can be understood that during the operation of the incubator, there may be temperature instability caused by rapid heating, and the incubator easily has a secondary temperature decrease. Therefore, in the technical solution provided in the disclosed embodiment, after adjusting the internal temperature to the set temperature, the current internal temperature of the incubator can be obtained via a temperature sensor arranged on the surface of the incubator, and in a case where the internal temperature of the incubator decreases and the decreasing exceeds the preset threshold, the internal temperature of the incubator is re-adjusted to the set temperature of the incubator. Here, the operator can set the preset threshold based on the required temperature of microbial cultivation. In one example, the preset threshold can be 0.1° C. Furthermore, as requiring secondary temperature adjustment of the incubator, the internal temperature of the incubator can be re-adjusted to the set temperature of the incubator. In this way, it can effectively regulate the internal temperature of the incubator in a timely manner when there is a problem of secondary decrease in the incubator, and reduce the adverse impact of secondary decrease in internal temperature on the cultivation effect.

Optionally, the step of S54, in a case where the internal temperature of the incubator decreases and a decreasing magnitude exceeds a preset threshold, re-regulating the internal temperature to the set temperature of the incubator comprises:
determining a secondary heating power of the heating wire; and
controlling the heating wire to operate at the secondary heating power.

In the present solution, in a case where the internal temperature of the incubator decreases and the decreasing magnitude exceeds the preset threshold, the incubator can re-adjust the internal temperature to the set temperature of the incubator. Specifically, the secondary heating power of the heating wire can be determined. Furthermore, the incubator can control the heating wire to operate at the secondary heating power. Here, the secondary scale factor can be determined firstly, and after determining the secondary scale factor, the secondary heating power for secondary regulation of the internal temperature of the incubator can be determined. It can be understood that due to the second decrease in temperature, the temperature difference between the internal temperature after decreasing and the set temperature is relatively small. In one example, the secondary scale factor Qₘ can be set as 0.3. With this solution, the secondary heating power for regulating the internal temperature of the incubator was accurately determined, so as to control the heating wire to operate at the secondary heating power, in order to effectively solve the technical problem of secondary temperature decrease in the incubator.

Optionally, the secondary heating power is determined by the following formula:

$${P}_{2 n d} = {P}_{max} \times ( {Q}_{m} - b \times t )$$

where P_{2nd} is the secondary heating power, Pₘₐₓ is a maximum heating power of the incubator, Qₘ is the secondary scale factor, b is a secondary amplitude factor, and t is the heating cycle.

In the present solution, after determining the secondary scale factor, the step for calculating the target heating power can be determined by combining the secondary scale factor. That is, when the operator presets the secondary scale factor as 0.3, calculating the secondary heating power P_{2nd}=Pₘₐₓ×(0.3−b×t) is determined. Here, Pₘₐₓ is the maximum heating power of the incubator, of which value is determinate and related to the volume of the incubator. In one example, if the volume of the incubator is 168 L, the corresponding Pₘₐₓ is 800 watts. The letter t represents the heating cycle, which can be preset by the operator, where the heating wire has the same heating power within the same heating cycle. The letter b is the secondary amplitude factor, here, the temperature rise amplitude during the secondary temperature adjustment is slightly lower than the temperature rise amplitude during the primary temperature adjustment. The amplitude factor is inversely proportional to the rise amplitude situation. Therefore, the secondary amplitude factor b is slightly higher than the amplitude factor a. Here, if the amplitude factor a is 0.02, then the secondary amplitude factor can be 0.04. With this solution, the secondary heating power for regulating the internal temperature of the incubator can be more accurately determined, and an accurate data basis can be provided for the heating control of the heating wire.

In the actual implement, when the door body is opened and then reclosed by the operator, the incubator may determine the set temperature of the incubator through the display information on the incubator panel, and determine the target scale factor corresponding to the temperature range according to the temperature range corresponding to the set temperature. In this way, the target heating power used to regulate the internal temperature of the incubator is determined. Furthermore, if the multiple inner surfaces of the incubator are arranged with the heating wires, the heating power corresponding to the heating wires on each inner surface can be determined by combining the correspondence between the predetermined target heating power and the heating power of multiple inner surfaces. And the heating wires arranged on multiple internal surfaces of the incubator are controlled to operate at the heating power corresponding to each heating wire, to adjust the current internal temperature of the incubator to the set temperature. If the current internal temperature of the incubator continues to decrease and the decrease exceeds the threshold after a period of operation, the internal temperature of the incubator can be adjusted based on the determined secondary heating power to avoid the impact of secondary temperature decrease on the cultivation effect.

A device for regulating the temperature of an incubator provided by the embodiment of the present disclosure, comprises: an acquisition module, a determination module, and a control module. The acquisition module is configured to obtain a set temperature of the incubator in a case where a door body of the incubator is opened and then reclosed; the determination module is configured to determine a target heating power according to the set temperature; and the control module is configured to control the heating wire to operate at the target heating power so as to adjust the internal temperature of the incubator to the set temperature.

With the device for regulating the temperature of an incubator provided by the embodiment of the present disclosure, obtaining a set temperature of the incubator in a case where the door body of the incubator is opened and then reclosed, combining a heating power corresponding to the set temperature or a temperature range corresponding to the set temperature, and determining a target heating power used to regulate the internal temperature of the incubator, in the situation of opening and closing the door of the incubator, the heating wire of the incubator is controlled to operate at the determined target heating power, which replaces the control of the heating wire by one or more sets of PID control algorithms. By means of mandatory intervention, the internal temperature of the incubator is rapidly restored to the set temperature of the incubator, so as to reduce the adverse impact of door opening and closing operations on the cultivation effect of the incubator.

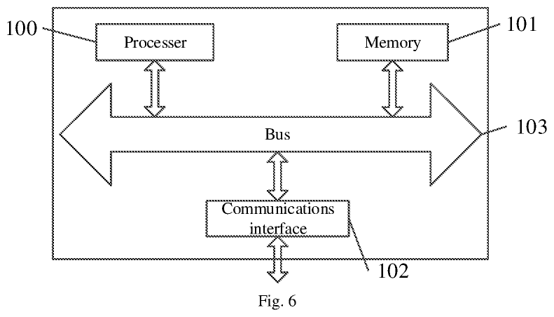

FIG. 6 is a schematic diagram of a device for regulating the temperature of an incubator provided by an embodiment of the present disclosure. With reference to FIG. 6, the embodiment of the present disclosure provides a device for regulating the temperature of an incubator, which comprises a processor 100 and a memory 101. Optionally, the device may also include a communication interface 102 and a bus 103. The processor 100, the communication interface 102, and the memory 101 are communicated with each other via the bus 103. The communication interface 102 may be configured for information transmission. The processor 100 may invoke logic instructions in the memory 101 to execute the method for regulating the temperature of the incubator in the mentioned embodiment.

Further, the logic instructions in the memory 101 described above may be realized in the form of software functional units and may be stored in a computer-readable storage medium when sold or used as a separate product.

As a computer-readable storage medium, the memory 101 may be configured to store software programs, and computer executable programs, such as program instructions/modules corresponding to the methods in embodiments of the present disclosure. The processor 100 executes the function application and data processing by running the program instructions/modules stored in the memory 101 that is to implement the method for regulating the temperature of the incubator in the mentioned embodiment.

The memory 101 may comprise a stored program area and a stored data area, wherein the stored program area may store an operating system and an application program required for at least one function. The storage data area may store data created according to the use of the terminal device. In addition, the memory 101 may include a high-speed random access memory and may also include a non-volatile memory.

The embodiment of the present disclosure provides an incubator comprising: the mentioned device for regulating the temperature of the incubator.

The embodiments of the present disclosure provide a computer-readable storage medium storing computer-executable instructions configured to execute the above-mentioned method for regulating the temperature of the incubator.

The embodiments of the present disclosure provide a computer program product, which comprises a computer program stored on a computer-readable storage medium. The computer program includes program instructions that, when executed by a computer, cause the computer to execute the method for regulating the temperature of the incubator.

The computer-readable storage medium may be a transient computer-readable storage medium or a non-transient computer-readable storage medium.

The technical solution of the embodiment of the present disclosure can be embodied in the form of a software product, The computer software product is stored in a storage medium and includes one or more instructions for causing a computer device (which may be a personal computer, server, or network device, etc.) to perform all or part of the steps of the method described in the embodiment of the present disclosure. The above-mentioned storage medium may be a non-transient storage medium, including a USB disk, a mobile hard disk, a Read-Only Memory (ROM), a Random Access Memory (RAM), a magnetic disk or an optical disk, and other media capable of storing program codes or maybe a transient storage medium.

The above description and drawings sufficiently illustrate embodiments of the present disclosure to enable to practice by those skilled in the art. Other embodiments may include structural logical electrical procedural and other modifications. Embodiments represent only possible variations. Unless explicitly required, individual parts and functions are optional, and the order of operation can vary. Portions and features of some embodiments may be included in or in place of portions and features of other embodiments. Furthermore, the terms used in the present disclosure are used only to describe embodiments and are not used to limit the claims. As used in the embodiments and the description of the claims, the singular forms of “a”, “an” and “the” are intended to include the plural forms as well, unless the context clearly indicates. Similarly, the term “and/or” as used in this application means encompassing one or more associated lists of any and all possible combinations. Additionally, when used in this application, the term “comprise” and its variants “comprises” and/or “comprising”, etc. refer to the presence of stated features, totals, steps, operations, elements, and/or components, but do not exclude the presence or addition of one or more other features, totals, steps, operations, elements, components, and/or groupings of these. In the absence of further limitations, an element defined by the phrase “comprises a/an . . . ” does not preclude the existence of another identical element in the process, method, or device in which the element is included. Herein each embodiment may be highlighted as being different from the other embodiments and the same similar parts between the various embodiments may be referred to with respect to each other. For the method, product, etc. disclosed by the embodiment, if it corresponds to the method portion disclosed by the embodiment, reference can be made to the description of the method portion where relevant.

Those skilled in the art will appreciate that the various example units and algorithm steps described in connection with the embodiments disclosed herein can be implemented in electronic hardware or a combination of computer software and electronic hardware. Whether these functions are performed in hardware or software can depend on the specific application and design constraints of the technical solution. The skilled artisan may use different methods for each particular application to implement the described functionality but such implementation should not be considered outside the scope of the disclosed embodiments. It will be apparent to the skilled person that for convenience and conciseness of description, the specific operating processes of the above-mentioned systems, devices, and units may be referred to the corresponding processes in the mentioned method embodiments and will not be repeated herein.

In the embodiments disclosed herein, the disclosed methods, and products (including but not limited to devices, devices, etc.) may be implemented in other ways. For example, the above-mentioned embodiment of the device is only schematic, for example, the division of the unit may be only a logical function division, and in practice, there may be another division mode, for example, multiple units or components may be combined or integrated into another system, or some features may be ignored or not performed. In addition, the coupling or direct coupling or communication connection between each other shown or discussed may be indirect coupling or communication connection through some interface, device, or unit, and may be electrical, mechanical, or another form. The elements illustrated as separate elements may or may not be physically separated, and the elements displayed as elements may or may not be physical elements, i.e. may be located in one place or may be distributed over a plurality of network elements. Some or all of the units can be selected according to actual needs to realize the embodiment. In addition, each functional unit in the embodiment of the present disclosure may be integrated into one processing unit, each unit may exist physically alone, or two or more units may be integrated into one unit.

The flowcharts and block diagrams in the accompanying drawings illustrate the architecture functionality and operation of possible implementations of systems methods and computer program products according to embodiments of the present disclosure. In this regard, each block in a flow chart or block diagram may represent a module, program segment, or part of code containing one or more executable instructions for performing a specified logical function. In some alternative implementations, the functions indicated in the boxes may also occur in a different order than those indicated in the drawings. For example, two successive boxes can actually be executed substantially in parallel, or they can sometimes be executed in reverse order, depending on the functionality involved. In the description corresponding to the flowcharts and block diagrams in the drawings, the operations or steps corresponding to different blocks may also occur in a different order than that disclosed in the description, and sometimes there is no specific order between the different operations or steps. For example, two successive operations or steps can actually be performed substantially in parallel, or they can sometimes be performed in reverse order, depending on the functionality involved. Each block in the block diagram and/or flow chart, and a combination of the blocks in the block diagram and/or flow chart, may be implemented in a dedicated hardware-based system that performs a specified function or action or may be implemented in a combination of dedicated hardware and computer instructions.

1. A method for regulating a temperature of an incubator, an inner surface of the incubator being arranged with heating wires, the method comprising:
in case where a door body of the incubator is opened and then reclosed, obtaining a set temperature of the incubator;
according to at least one of a heating power corresponding to the set temperature and a temperature range corresponding to the set temperature, determining a target heating power; and
controlling the heating wires to operate at the target heating power to adjust an internal temperature of the incubator to the set temperature.

2. The method according to claim 1, wherein the according to at least one of a heating power corresponding to the set temperature and a temperature range corresponding to the set temperature, determining the target heating power comprises:
according to a preset first correspondence, defining the heating power corresponding to the set temperature as the target heating power.

3. The method according to claim 1, wherein the according to at least one of a heating power corresponding to the set temperature and a temperature range corresponding to the set temperature, determining the target heating power comprises:
according to a preset second correspondence, determining a target scale factor corresponding to the temperature range; and
according to the target scale factor, calculating the target heating power.

4. The method according to claim 3, wherein the according to the target scale factor, calculating the target heating power comprises:
$${P}_{target} = {P}_{max} \times ( {Q}_{n} - a \times t )$$
wherein P_{target} is the target heating power, Pₘₐₓ is a maximum heating power of the incubator, Qₙ is the target scale factor, a is an amplitude factor, and t is a heating cycle.

5. The method according to claim 1, wherein the in case where the door body of the incubator is opened and then reclosed, obtaining a set temperature of the incubator comprises:
in case where the door body of the incubator is opened and then reclosed, obtaining a temperature change rate inside the incubator; and
in case where the temperature change rate inside the incubator is greater than or equal to a preset change rate, obtaining the set temperature of the incubator.

6. The method according to claim 1, after adjusting the internal temperature of the incubator to the set temperature, further comprising:
in case where the internal temperature of the incubator decreases and a decreasing magnitude exceeds a preset threshold, re-regulating the internal temperature of the incubator to the set temperature of the incubator.

7. The method according to claim 6, after adjusting the internal temperature of the incubator to the set temperature of the incubator, further comprising:
determining the secondary heating power of the heating wires; and
controlling the heating wires to operate at the secondary heating power.

8. The method according to claim 7, wherein the secondary heating power is determined by the following formula:
$${P}_{2 n d} = {P}_{max} \times ( {Q}_{m} - b \times t )$$
wherein P_{2nd} is the secondary heating power, Pₘₐₓ is a maximum heating power of the incubator, Qₘ is the secondary scale factor, b is a secondary amplitude factor, and t is a heating cycle.

9. A device for regulating a temperature of an incubator, comprising a processor and a memory storing program instructions, wherein
the processor is configured to execute the steps of:
in case where a door body of the incubator is opened and then reclosed, obtaining a set temperature of the incubator;
according to at least one of a heating power corresponding to the set temperature and a temperature range corresponding to the set temperature, determining a target heating power; and
controlling the heating wires to operate at the target heating power to adjust the internal temperature of the incubator to the set temperature.

10. The device according to claim 9, further mounted on the incubator, an inner surface of the incubator being arranged with heating wires.

11. The method according to claim 2, after adjusting the internal temperature of the incubator to the set temperature, further comprising:
in case where the internal temperature of the incubator decreases and a decreasing magnitude exceeds a preset threshold, re-regulating the internal temperature of the incubator to the set temperature of the incubator.

12. The method according to claim 11, after adjusting the internal temperature of the incubator to the set temperature of the incubator, further comprising:
determining the secondary heating power of the heating wires; and
controlling the heating wires to operate at the secondary heating power.

13. The method according to claim 12, wherein the secondary heating power is determined by the following formula:
$${P}_{2 nd} = {P}_{max} \times ( {Q}_{m} - b \times t )$$
wherein P_{2nd} is the secondary heating power, Pₘₐₓ is a maximum heating power of the incubator, Qₘ is the secondary scale factor, b is a secondary amplitude factor, and t is a heating cycle.

14. The method according to claim 3, after adjusting the internal temperature of the incubator to the set temperature, further comprising:
in case where the internal temperature of the incubator decreases and a decreasing magnitude exceeds a preset threshold, re-regulating the internal temperature of the incubator to the set temperature of the incubator.

15. The method according to claim 14, after adjusting the internal temperature of the incubator to the set temperature of the incubator, further comprising:
determining the secondary heating power of the heating wires; and
controlling the heating wires to operate at the secondary heating power.

16. The method according to claim 15, wherein the secondary heating power is determined by the following formula:
$${P}_{2 n d} = {P}_{max} \times ( {Q}_{m} - b \times t )$$
wherein P_{2nd} is the secondary heating power, Pₘₐₓ is a maximum heating power of the incubator, Qₘ is the secondary scale factor, b is a secondary amplitude factor, and t is a heating cycle.

17. The method according to claim 4, after adjusting the internal temperature of the incubator to the set temperature, further comprising:
in case where the internal temperature of the incubator decreases and a decreasing magnitude exceeds a preset threshold, re-regulating the internal temperature of the incubator to the set temperature of the incubator.

18. The method according to claim 17, after adjusting the internal temperature of the incubator to the set temperature of the incubator, further comprising:
determining the secondary heating power of the heating wires; and
controlling the heating wires to operate at the secondary heating power.

19. The method according to claim 18, wherein the secondary heating power is determined by the following formula:
$${P}_{2 n d} = {P}_{max} \times ( {Q}_{m} - b \times t )$$
wherein P_{2nd} is the secondary heating power, Pₘₐₓ is a maximum heating power of the incubator, Qₘ is the secondary scale factor, b is a secondary amplitude factor, and t is a heating cycle.

20. The method according to claim 5, after adjusting the internal temperature of the incubator to the set temperature, further comprising:
in case where the internal temperature of the incubator decreases and a decreasing magnitude exceeds a preset threshold, re-regulating the internal temperature of the incubator to the set temperature of the incubator.
